(19) United States
(12) Patent Application Publication
(10) Pub. No.: US 2020/0222571 A1
(43) Pub. Date: Jul. 16, 2020

(54) AROMATHERAPY DEVICE

(71) Applicant: DONGGUAN YIH TEH ELECTRIC PRODUCTS CO., LTD., Dongguan (CN)

(72) Inventor: Ming Jen HSIAO, Miaoli County (TW)

(21) Appl. No.: 16/828,043

(22) Filed: Mar. 24, 2020

Related U.S. Application Data
(63) Continuation-in-part of application No. 15/884,346, filed on Jan. 30, 2018.
Application No. 15/884,346 is a continuation-in-part of application No. 15/862,356, filed on Jan. 4, 2018.
Application No. 15/862,356 is a continuation-in-part of application No. 15/653,362, filed on Jul. 18, 2017, now abandoned.
Application No. 15/884,346 is a continuation-in-part of application No. 15/653,362, filed on Jul. 18, 2017, now abandoned.
Application No. 15/862,356 is a continuation-in-part of application No. 14/938,564, filed on Nov. 11, 2015, now abandoned.
Application No. 14/938,564 is a continuation-in-part of application No. 15/258,757, filed on Sep. 7, 2016, now Pat. No. 10,064,969.
Application No. 15/258,757 is a continuation-in-part of application No. 15/887,232, filed on Feb. 2, 2018.
Application No. 15/887,232 is a continuation-in-part of application No. 15/866,181, filed on Jan. 9, 2018.
Application No. 15/866,181 is a continuation-in-part of application No. 15/653,362, filed on Jul. 18, 2017, now abandoned.
Application No. 15/653,362 is a continuation-in-part of application No. 15/258,757, filed on Sep. 7, 2016, now Pat. No. 10,064,969.
Application No. 15/653,362 is a continuation-in-part of application No. 15/258,757, filed on Sep. 7, 2016, now Pat. No. 10,064,969.
Application No. 15/258,757 is a continuation-in-part of application No. 15/258,689, filed on Sep. 7, 2016, now Pat. No. 9,844,609.
Application No. 15/258,689 is a continuation-in-part of application No. 14/157,302, filed on Jan. 16, 2014, now Pat. No. 9,500,358.
Application No. 14/157,302 is a continuation-in-part of application No. 14/042,162, filed on Sep. 30, 2013, now Pat. No. 9,498,553.
Application No. 14/042,162 is a continuation-in-part of application No. 13/670,430, filed on Nov. 6, 2012, now Pat. No. 8,938,159.
Application No. 13/670,430 is a continuation-in-part of application No. 13/669,354, filed on Nov. 5, 2012, now Pat. No. 9,109,780.
Application No. 13/669,354 is a continuation-in-part of application No. 13/549,493, filed on Jul. 15, 2012, now abandoned.
Application No. 13/549,493 is a continuation-in-part of application No. 13/658,820, filed on Oct. 24, 2012, now abandoned.
Application No. 13/658,820 is a continuation-in-part of application No. 13/549,490, filed on Jul. 15, 2012, now Pat. No. 8,668,885.
Application No. 15/258,757 is a continuation-in-part of application No. 14/157,302, filed on Jan. 16, 2014, now Pat. No. 9,500,358.
Application No. 14/157,302 is a continuation-in-part of application No. 14/042,162, filed on Sep. 30, 2013, now Pat. No. 9,498,553.
Application No. 14/042,162 is a continuation-in-part of application No. 13/670,430, filed on Nov. 6, 2012, now Pat. No. 8,938,159.
Application No. 13/670,430 is a continuation-in-part of application No. 13/549,490, filed on Jul. 15, 2012, now Pat. No. 8,668,885.
Application No. 13/549,490 is a continuation-in-part of application No. 13/549,493, filed on Jul. 15, 2012, now abandoned.
Application No. 13/549,493 is a continuation-in-part of application No. 13/658,820, filed on Oct. 24, 2012, now abandoned.
Application No. 13/658,820 is a continuation-in-part of application No. 13/669,354, filed on Nov. 5, 2012, now Pat. No. 9,109,780.
Application No. 14/157,302 is a continuation-in-part of application No. 13/670,430, filed on Nov. 6, 2012, now Pat. No. 8,938,159.
Application No. 13/670,430 is a continuation-in-part of application No. 13/669,354, filed on Nov. 5, 2012, now Pat. No. 9,109,780.
Application No. 13/669,354 is a continuation-in-part of application No. 13/658,820, filed on Oct. 24, 2012, now abandoned.
Application No. 14/042,162 is a continuation-in-part of application No. 13/658,820, filed on Oct. 24, 2012, now abandoned.
Application No. 13/658,820 is a continuation-in-part of application No. 13/549,490, filed on Jul. 15, 2012, now Pat. No. 8,668,885.
Application No. 13/549,490 is a continuation-in-part of application No. 13/549,493, filed on Jul. 15, 2012, now abandoned.

(51) Int. Cl. A61L 9/03; A61M 21/00

(52) U.S. Cl. CPC A61L 9/03; A61M 21/00; A61L 2209/12; A61M 2205/3653; A61L 2209/131; A61M 2021/0016; A61L 2209/133

(57) ABSTRACT

An aromatherapy device includes an aroma diffuser using a hollow heating element to heat thermal conduction device that is used to heat a disposable multilayer breathing film-based aroma capsule or aromatic substances in a detachable aromatic substance container for producing fragrance. The hollow heating element produces heat energy to the thermal conduction device without causing concentration of heat source, so that the thermal conduction device can evenly heat the disposable multilayer breathing film-based aroma capsule or the aromatic substances in the detachable aromatic substance container for producing fragrance in the space, enabling the aromatic substances to be completely vaporized without leaving a residue.

This application is a Continuation-in-Part of application Ser. No. 15/884,346, filed Jan. 30, 2018 and Ser. No. 15/887,232, filed Feb. 2, 2018.

### BACKGROUND OF THE INVENTION

### 1. Field of the Invention

The present invention relates to scent releasing and heating devices and more particularly, to an aromatherapy device with an aroma diffuser, which allows a disposable multilayer breathing film-based aroma capsule and a detachable aromatic substance container to be used interchangeably according to user preferences, so the multiple choices of aromatic substances are not limited.

### 2. Description of the Related Art

In prior art aroma diffuser designs invented by the present inventor, such as U.S. Pat. Nos. 8,668,885, 9,550,358, 9,498,533, EP Pat. No. 2679249, a container is used to transmit a heat source to an aromatic substance. The aromatic substance is heated in the middle and the other part of the aromatic substance is far away from the heat source. The fragrance is only generated in the middle part of the aromatic substance, so it is less easy to produce a sufficient amount of fragrance instantly, and you need to wait a long time to smell the fragrance. And the heat source is concentrated in the central part of the aromatic substance, it is difficult to completely vaporize the aromatic substance, and the residual aromatic substance is often formed to waste.

In the existing aroma diffuser designs, the heating container that houses the aromatic substance is a fixed structure. For example, it is a metal container fixed to the aroma diffuser. The interior of the metal container is filled with an aromatic substance, so the use of aromatic substances and the replacement of aromatic substances are limited.

### SUMMARY OF THE INVENTION

The present invention has been accomplished under the circumstances in view. It is thereof the main object of the present invention to provide an aromatherapy device with an aroma diffuser, which evenly disperses heat source through the aromatic substances for producing fragrance, enabling the aromatic substances to be completely vaporized without leaving a residue.

It is another object of the present invention to provide an aromatherapy device with an aroma diffuser, which allows a disposable multilayer breathing film-based aroma capsule and a detachable aromatic substance container to be used interchangeably according to user preferences, so the multiple choices of aromatic substances are not limited.

To achieve these and other objects of the present invention, an aromatherapy device comprises an aroma diffuser. The aroma diffuser comprises a connection device, a fixing device set, a hollow heating element, a thermal conduction device, a shell and a base. The shell has a top opening and an opposing bottom opening. The base is fastened to the bottom opening of the shell. The connection device is mounted inside the shell and has one end thereof facing toward the top opening and fastened to the bottom center of the thermal conduction device and an opposing end thereof connected to the base inside the shell. The hollow heating element surrounds the connection device. The fixing device set supports the bottom side of the hollow heating element. The hollow heating element has an opposing top side abutted against the bottom side of the thermal conduction device. The thermal conduction device of the aroma diffuser of the aromatherapy device can heat a disposable multilayer breathing film-based aroma capsule thereon. The hollow heating element is electrically connected to a power source to generate heat for heating the thermal conduction device. The structure of the thermal conduction device does not concentrate the heat source, and it can evenly heat the aroma capsule to quickly generate fragrance in the space and completely heat and vaporize the aromatic substance in the aroma capsule without leaving a residue.

Furthermore, an aromatic substance container can be used to replace the multilayer breathing film-based aroma capsule. The user can fill the desired aromatic substances into the aromatic substance container for heating by the thermal conduction device to produce fragrance. These aromatic substances can be solid or liquid aromatic substances with special flavors, home-made aromatic waxes, essential oils, dried flowers and herbs, or fragrant wood.

the hollow heating element 16 is an annular heating element. the hollow heating element 16 can distribute the heat source between the center and surrounding of the thermal conduction device 17. The feature of the hollow heating element 16 is that instead of focusing the heat source in the center, it will let the heat source be scattered around. In this way, the hollow heating element 16 can heat the thermal conduction device 17 evenly so that the thermal conduction device 17 can evenly transfer heat to the aroma capsule 12 or the aromatic substances in the aromatic substance container 11 to completely heat and vaporize the aromatic substances.

The beneficial effects of the present invention are outlined hereinafter, the hollow heating element can distribute the heat source between the center and surrounding of the thermal conduction device. The feature of the hollow heating element is that instead of focusing the heat source in the center, it will let the heat source be scattered around. In this way, the hollow heating element can heat the thermal conduction device evenly so that the thermal conduction device can evenly transfer heat to the aroma capsule or the aromatic substances in the aromatic substance container to completely heat and vaporize the aromatic substances without leaving a residue in the aroma capsule or the aromatic substance container. The invention eliminates the drawbacks of limited heat source power and poor heating of aromatic substances of the conventional aroma diffusers. Conventional aroma diffusers are small tabletop or wall socket mount designs, and the heat source provided by the heating element is focused on the center of the aromatic substance. Due to the limitation of the small aroma diffuser internal space and the small heat source power, the aromatic substance heating effect of the conventional aroma diffusers is poor. After the aroma diffuser is powered on, you also need to wait for a long time before there is enough fragrance to spread in the space. The hollow heating element of the aroma diffuser of the present invention disperse the heat source to evenly heat the aromatic substance, improving the problem that the heat source of the conventional heating element is concentrated in the aromatic substance. At the same time, the aroma diffuser of the present invention can also rapidly generate an aromatic odor in the air by using a uniformly dispersed heat source at a safe low heating temperature.

### BRIEF DESCRIPTION OF THE DRAWINGS

FIG. 1 is an oblique top elevational view of an aromatherapy device in accordance with the present invention.

FIG. 2 is an exploded view of the aroma diffuser using a multilayer breathing film-based aroma capsule of the aromatherapy device in accordance with the present invention.

FIG. 3 is an exploded view of the aroma diffuser using a detachable aromatic substance container of the aromatherapy device in accordance with the present invention.

FIG. 4 is a longitudinal sectional view of the aromatherapy device shown in FIG. 1.

FIG. 5 an oblique top elevational view of an alternate form of the aromatherapy device in accordance with the present invention.

FIG. 6 is an exploded view of the aroma diffuser using a multilayer breathing film-based aroma capsule of the aromatherapy device shown in FIG. 5.

FIG. 7 is an exploded view of the aroma diffuser using a detachable aromatic substance container of the aromatherapy device shown in FIG. 5.

FIG. 8 is a longitudinal sectional view of the aromatherapy device shown in FIG. 5.

FIG. 9 is a sectional exploded elevation of the multilayer breathing film-based aroma capsule.

### DETAILED DESCRIPTION OF THE PREFERRED EMBODIMENT

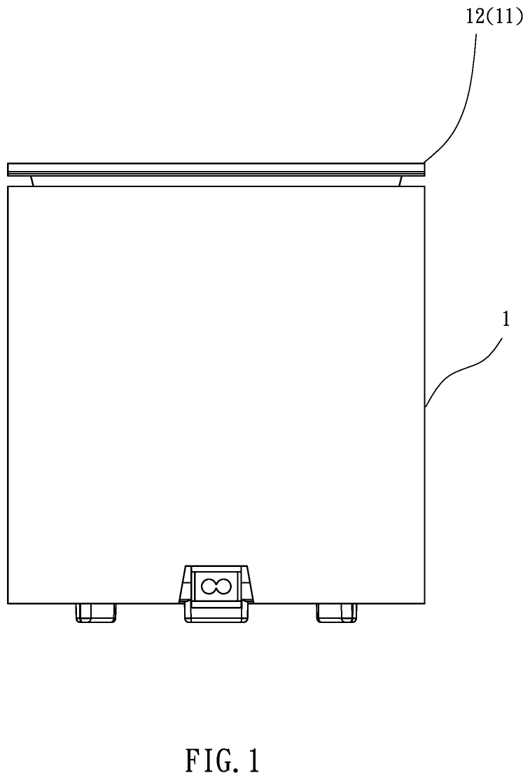

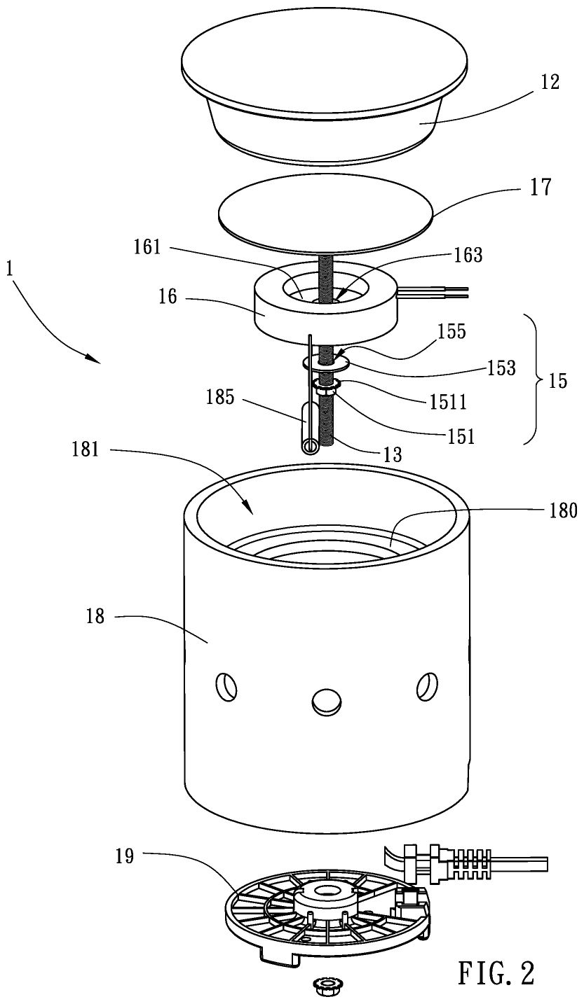

Referring to FIGS. 1 and 2, an aromatherapy device includes an aroma diffuser 1. The aroma diffuser 1 comprises a connection device 13, a fixing device set 15, a hollow heating element 16, a thermal conduction device 17, a shell 18, and a base 19. The shell 18 has a top opening 181 and an opposing bottom opening 183. The base 19 is fastened to the bottom opening 183. The connection device 13 is mounted in the shell 18, having one end thereof facing toward the top opening 181 and fastened to the bottom center of the thermal conduction device 17 and an opposing end thereof affixed to the base 19 inside the shell 18. The hollow heating element 16 surrounds the connection device 13. The fixing device set 15 supports the bottom side of the hollow heating element 16. The opposing top side of the hollow heating element 16 abuts against the bottom side of the thermal conduction device 17. The opposing top side of the thermal conduction device 17 is used to heat a multilayer breathing film-based aroma capsule 12. The hollow heating element 16 is electrically connected to a power source to generate heat for heating the thermal conduction device 17. The structure of the thermal conduction device 17 does not concentrate the heat source, and it can evenly heat the aroma capsule 12 to quickly generate fragrance in the space and completely heat and vaporize the aromatic substance in the aroma capsule 12.

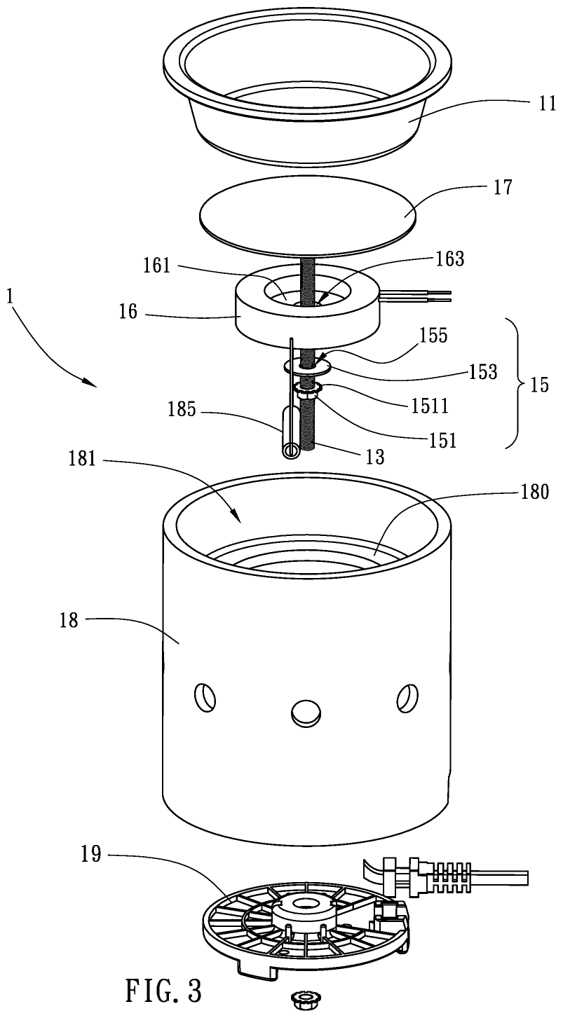

Referring to FIGS. 1 and 3, in an alternate form of the present invention, the aroma capsule 12 is replaced by an aromatic substance container 11. Thus, the user can fill external aromatic substances into the aromatic substance container 11 and heat it to generate fragrance. These aromatic substances can be solid or liquid aromatic substances with special flavors, or home-made aromatic waves, essential oil, dried flowers and herbs, fragrant wood and other various aromatic substances.

According to this alternate form, the aroma diffuser 1 of the aromatherapy device comprises a connection device 13, a fixing device set 15, a hollow heating element 16, a thermal conduction device 17, a shell 18, and a base 19. The shell 18 has a top opening 181 and an opposing bottom opening 183. The base 19 is fastened to the bottom opening 183. The connection device 13 is mounted in the shell 18, having one end thereof facing toward the top opening 181 and fastened to the bottom center of the thermal conduction device 17 and an opposing end thereof affixed to the base 19 inside the shell 18. The hollow heating element 16 surrounds the connection device 13. The fixing device set 15 supports the bottom side of the hollow heating element 16. The opposing top side of the hollow heating element 16 abuts against the bottom side of the thermal conduction device 17. The opposing top side of the thermal conduction device 17 is used to heat an aromatic substance container 11. Thus, the user can fill the desired aromatic substances into the aromatic substance container 11. The hollow heating element 16 is electrically connected to a power source to generate heat for heating the thermal conduction device 17. The structure of the thermal conduction device 17 does not concentrate the heat source, and it can evenly heat the aromatic substance container 11 to quickly generate fragrance in the space and completely heat and vaporize the aromatic substances in the aromatic substance container 11, avoiding waste of aromatic substances.

In some embodiments of the present invention, the hollow heating element 16 can be an annular heating element, a hollow plum-shaped heating element, a hollow rectangular heating element, a hollow triangular heating element, a hollow diamond-shaped heating element, or a hollow irregular ring-shaped heating element. In the embodiment shown in FIGS. 2 and 3, the hollow heating element 16 is an annular heating element. the hollow heating element 16 can distribute the heat source between the center and surrounding of the thermal conduction device 17. The feature of the hollow heating element 16 is that instead of focusing the heat source in the center, it will let the heat source be scattered around. In this way, the hollow heating element 16 can heat the thermal conduction device 17 evenly so that the thermal conduction device 17 can evenly transfer heat to the aroma capsule 12 or the aromatic substances in the aromatic substance container 11 to completely heat and vaporize the aromatic substances.

The invention eliminates the drawbacks of limited heat source power and poor heating of aromatic substances of the conventional aroma diffusers. Conventional aroma diffusers are small tabletop or wall socket mount designs, and the heat source provided by the heating element is focused on the center of the aromatic substance. Due to the limitation of the small aroma diffuser internal space and the small heat source power, the aromatic substance is heated below 90° C., normally in the range of 70˜80° C. or 80˜90° C. Thus, the aromatic substance heating effect of the conventional aroma diffusers is poor. After the aroma diffuser is powered on, you also need to wait for a long time before there is enough fragrance to spread in the space. The hollow heating element 16 of the aroma diffuser of the present invention disperse the heat source to evenly heat the aromatic substance, improving the problem that the heat source of the conventional heating element is concentrated in the aromatic substance. At the same time, the aroma diffuser of the present invention can also rapidly generate an aromatic odor in the air by using a uniformly dispersed heat source at a safe low heating temperature.

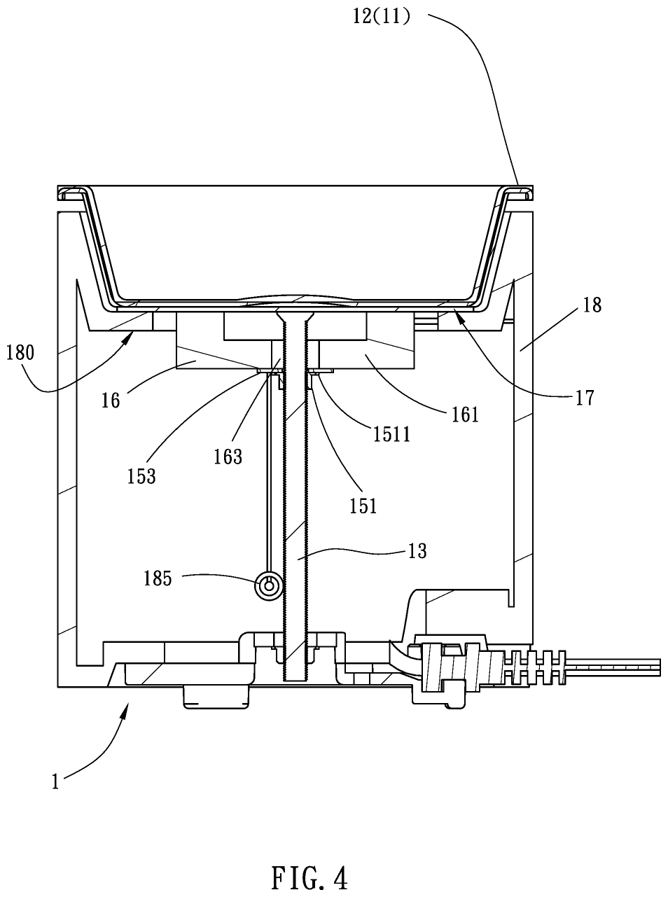

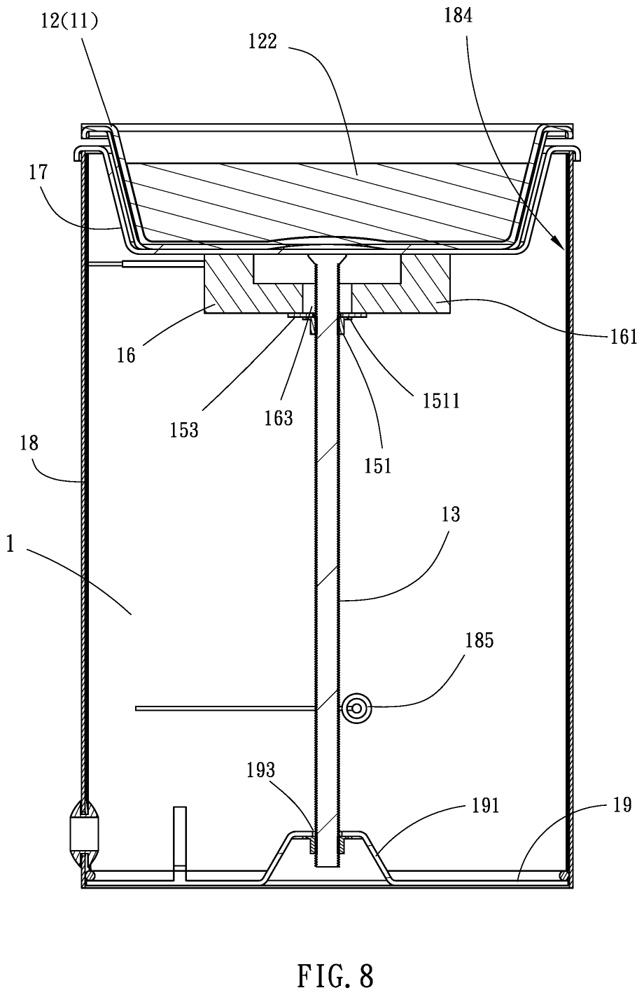

Referring to FIGS. 2, 3, 4 and 8, in one embodiment of the present invention, the hollow heating element 16 comprises an inner bottom flange 161 inwardly extended from a bottom side thereof, and a connection hole 163 surrounded by the inner bottom flange 161. The connection device 13 has its one end inserted through the connection hole 163 and fastened to the bottom center of the thermal conduction device 17. The inner bottom flange 161 increases the bottom surface area of the hollow heating element 16, thereby increasing the heating power of the hollow heating element 16 without changing the hollow heating shape on the upper side of the hollow heating element 16.

Referring to FIGS. 2, 4, 6 and 8, in one embodiment of the present invention, the thermal conduction device 17 or the aromatic substance container 11 is, for example, any one of metal, ceramic, glass, etc. in another embodiment of the present invention, the thermal conduction device 17 or the aromatic substance container 11 is, for example, iron, aluminum, copper, or an alloy made of metal, and has good thermal performance.

According to some embodiments of the present invention, the thermal conduction device 17 includes, for example, a metal sheet or a ceramic sheet.

In the embodiment shown in FIGS. 2-4, the thermal conduction device 17 includes a metal sheet, and the metal sheet can be, for example, an iron sheet.

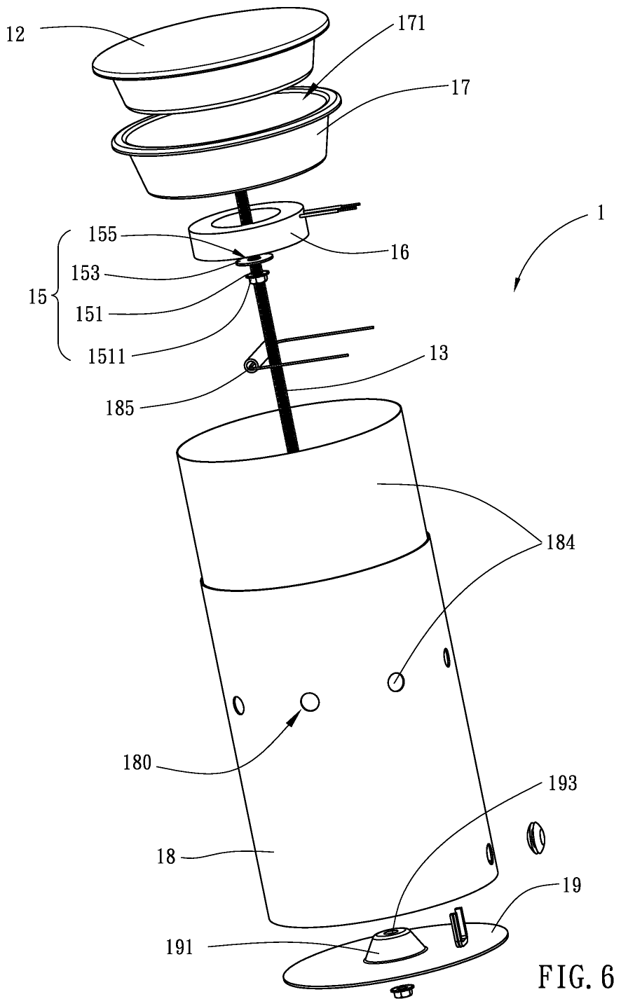

Referring to FIGS. 6-8, in some embodiments of the present invention, the thermal conduction device 17 can be a metal container or a ceramic container. The metal container can be a thermal conductor of aluminum, copper or iron. The metal container of the thermal conduction device 17 defines therein an accommodation chamber 171 for holding a multilayer breathing film-based aroma capsule 12 or an aromatic substance container 11. The metal container of the thermal conduction device 17 has the bottom side thereof disposed in contact with the hollow heating element 16. In this way, the metal container of the thermal conduction device 17 is used to heat the aroma capsule 12 or the aromatic substance in the aromatic substance container 11 to generate fragrance. The aroma capsule 12 or the aromatic substance container 11 can be used interchangeably according to user preferences, so the multiple choices of aromatic substances are not limited.

The aroma capsule 12 or the aromatic substance container 11 can be detachably attached to the top side of the metal sheet of the thermal conduction device 17, so that the heat source can be transferred to the aromatic substance in the aroma capsule 12 or the aromatic substance container 11, causing generation of fragrance. The user can selectively uses the disposable aroma capsule 12 or the detachable metal or ceramic aromatic substance container 11. When the aromatic substance container 11 is selected, the user can fill different aromatic substances in the aromatic substance container 11. The aromatherapy device of the present invention highlights the convenience and selectivity of the use of aromatic substances and solves the problem of the limitation of the function of conventional aroma diffusers.

Referring to FIGS. 2, 3, 6 and 7, in one embodiment of the present invention, the shell 18 is selected from light-transmissive or semi-light-transmissive materials, such as light-transmissive ceramic, plastic, glass, silicone, or the shell 18 can be provided with light holes. The light-transmissive or semi-light-transmissive shell 18 has an inner flange 180 formed on the inner side thereof. The thermal conduction device 17 is mounted on the inner flange 180 of the shell 18. The aromatherapy device further comprises a light-emitting device 185 mounted inside the shell 18 of the aroma diffuser 1. The light-emitting device 185 is, for example, a light source such as an LED or a light bulb connected to a power source to emit light radiation through the shell 18 to produce a lighting effect.

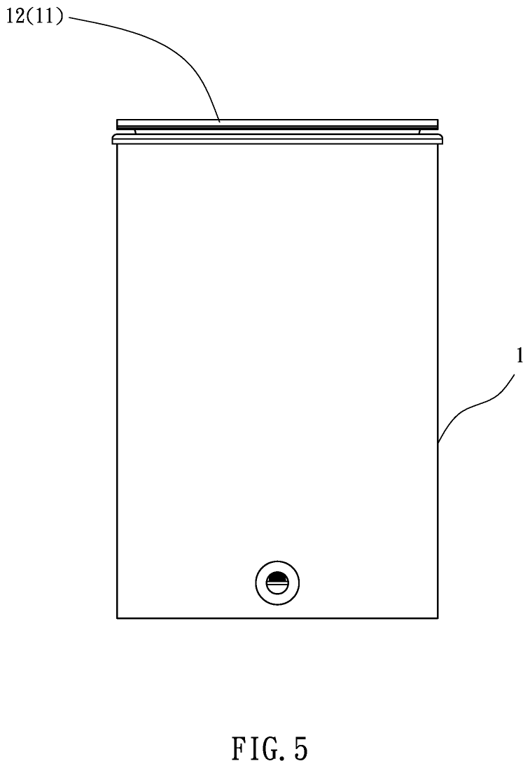

Referring to FIGS. 5-8, in one embodiment of the present invention, the material of the shell 18 is metal, ceramic or plastic. The base 19 has a stud 191. The stud 191 has a hole 193 in the center. The other end of the connection device 13 is fastened to the hole 193. In this embodiment, the shell 18 has a plurality of light holes 182. The light-transmissive shell 18 is mounted with a light-emitting device 185. The light-emitting device 185 can be LED or lamp bulb electrically connected to an external power source for emitting light through the light holes 182 to produce a decorative lighting effect.

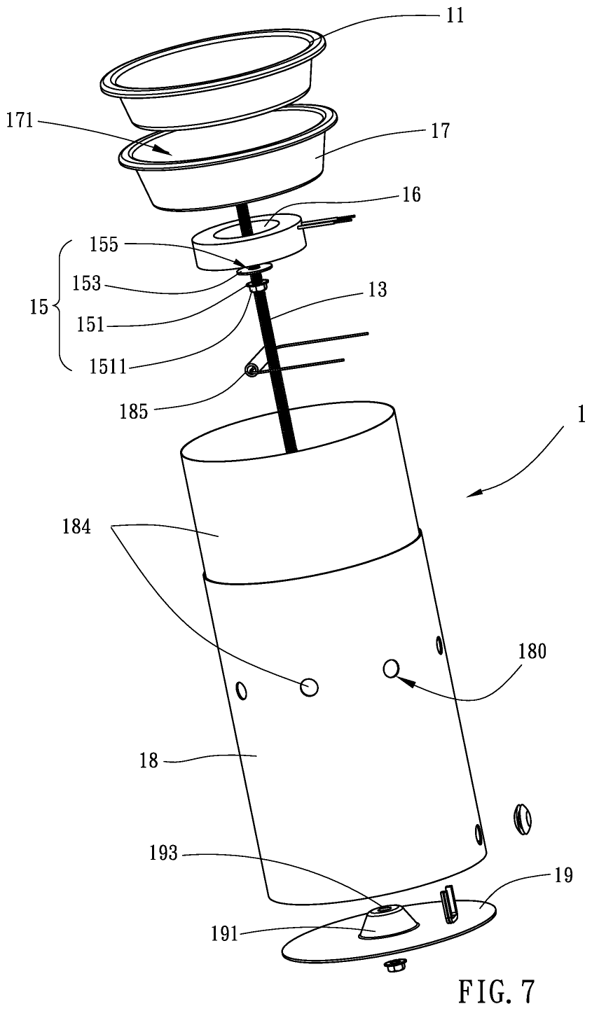

Referring to FIGS. 6 and 7, in one embodiment of the present invention, the metal shell 18 is provided with a diffuser 184 for creating a halo effect. The diffuser 184 is attached to the light holes 182. As shown in the drawings, the diffuser 184 is attached to the inner surface of the shell 18 to diffuse the light emitted by the light-emitting device 185 through the light holes 182, thereby creating a halo effect. The diffuser 184 includes, for example, translucent PP plastic diffuser, silicone, colored plastic film or translucent glass.

Referring to FIGS. 2-4 and FIGS. 6-8, in some embodiments of the present invention, the connection device 13 is, for example, a screw rod. The fixing device set 15 comprises a anti-loosening nut 151 and a washer 153. The washer 153 defines therein a hole 155. The hole 155 is attached onto the screw rod of the connection device 13. The anti-loosening nut 151 has friction spines 1511 located on a top side thereof. The anti-loosening nut 151 is threaded onto the screw rod of the connection device 13 and stopped against the washer 153. In this way, the friction spines 1511 can be used to increase the friction of the washer 153 to prevent the heat source from expanding and contracting the anti-loosening nut 151. Thus, the anti-loosening nut 151 maintains the washer 153 firmly against the hollow heating element 16, keeping the hollow heating element 16 in close contact with the thermal conduction device 17.

The aroma capsule 12 is placed on and heated by thermal conduction device 17.

Referring to FIGS. 2, 6, 8 and 9, in one embodiment of the present invention, the aroma capsule 12 comprises a heat-transfer container 120 that defines a top opening 1200, an aromatic substance 122 held in the heat-transfer container 120, and a breathing film 126 bonded to the heat-transfer container 120 over the top opening 1200 and the aromatic substance 122 to allow moisture vapor to be transmitted through the material.

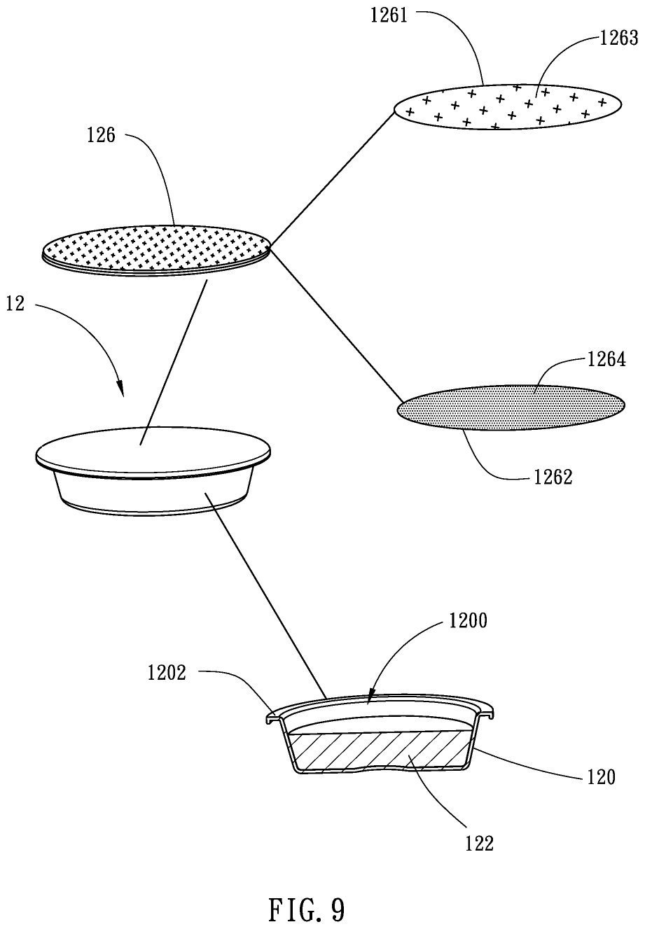

Referring to FIG. 9, in one embodiment of the present invention, the breathing film 126 comprises a fiber fixation layer 1261 and a microporous layer 1262 of excellent moisture permeability. The fiber fixation layer 1261 defines therein multiple first pores 1263. The microporous layer 1262 defines therein multiple second pores 1264. The fiber fixation layer 1261 and the microporous layer 1262 are bonded together to form the breathing film 126 with the breathable and waterproof function. The microporous layer 1262 is preferably made out of thermoplastic elastomer (TPE) compound. The fiber fixation layer 1261 is preferably made out of polyester. The polyester fiber fixation layer 1261 and the thermoplastic elastomer (TPE) microporous layer 1262 are bonded together to form the breathing film 126 that is breathable. The breathable aperture of the first pores 1263 is greater than the breathable aperture of the second pores 1264, facilitating outward transmittance of air. The breathing film 126 is bonded to the heat-transfer container 120 over the top opening 1200. Through the second pores 1264 of the thermoplastic elastomer (TPE) microporous layer 1262, the aroma capsule 12 provides waterproof and moisture transmissive functions, keeping the aromatic substance 122 fresh.

The fiber fixation layer 1261 does not shrink or expand the size and can keep the thermoplastic elastomer (TPE) microporous layer 1262 in shape, and thus, the surface of the breathing film 126 can be constantly maintained smooth without wrinkles. During the heating process, aromatic substance 122 keeps releasing aromatic vapor molecules that flow smoothly through the second pores 1264 of the thermoplastic elastomer (TPE) microporous layer 1262 and the first pores 1263 of the fiber fixation layer 1261 toward the atmosphere outside the aroma capsule 12, however, the liquid phase aromatic substance is prohibited from passing through the second pores 1264, ensuring safety application of the aroma capsule 12.

The aromatic substance 122 is selected from any one including, for example, balsam, spice, balm, sesame oil, fragrance blend, or essential oil.

After the aromatic substance 122 is used up, the heat-transfer container 120 can be thrown away or recycled, and a new aroma capsule 12 can be put in the thermal conduction device 17 of the aroma diffuser 1. Thus, the user's hand will not be contaminated by the aromatic substance 122. In addition, the user does not necessary to clean the thermal conduction device 17 of the aroma diffuser 1. In case the aroma diffuser 1 falls down accidentally during application, the melted aromatic substance 122 of the aroma capsule 12 is still held in the heat-transfer container 120 by the breathing film 126 and will not flow to the outside of the aroma diffuser 1 to cause contamination or dangers.

Referring to FIG. 9, in one embodiment of the present invention, the aroma capsule 12 further comprises a rim 1202 extended around the top opening 1200, and the breathing film 126 is bonded to the rim 1202. Preferably, the thermoplastic elastomer (TPE) microporous layer 1262 is peripherally bonded to the rim 1202 of the heat-transfer container 120 over the top opening 1200 to secure the breathing film 126 to the heat-transfer container 120.

In one embodiment of the present invention, the heat-transfer container 120 is a plastic heat-transfer container 120; the fiber fixation layer 1261 is made out of PET; the thermoplastic elastomer (TPE) microporous layer 1262 is made out of thermoplastic polyurethane (TPU); the PET fiber fixation layer 1261 and the thermoplastic elastomer (TPE) microporous layer 1262 are bonded together by hot press bonding or high-frequency sealing without affecting the breathing film 126; the TPU microporous layer 1262 of the breathing film 126 is peripherally bonded to the top opening 1200 of the plastic (such as PCTG) heat-transfer container 120 by hot press bonding or high-frequency sealing; the TPU microporous layer 1262 is peripherally bonded to the rim 1202 of the PCTG heat-transfer container 120 over the top opening 1200, and covered over the aromatic substance 122 to seal the aromatic substance 122 in the aroma capsule 12.

In one embodiment of the present invention, the plastic heat-transfer container 120 is made out of PCTG; the fiber fixation layer 1261 of the breathing film 126 is made out of PET; the microporous layer 1262 of the breathing film 126 is made out of TPU; subject to the fusible nature between PTU and PCTG, the TPU layer of the breathing film 126 is peripherally bonded to the rim 1202 of the PCTG heat-transfer container 120 around top opening 1200 by an automatic machine, improving the bonding quality and production rate and saving the cost. Further, an adhesive can be used to bond the breathing film 126 and the heat-transfer container 120 together.

Referring to FIG. 9, the fiber fixation layer 1261 and microporous layer 1262 of the aroma capsule 12 can be swapped upside down. In this case, the top side of the fiber fixation layer 1261 is bonded to the microporous layer 1262 to create the breathing film 126; the fiber fixation layer 1261 is peripherally bonded to the heat-transfer container 120 over the top opening 1200; when the aromatic substance 122 (such as aromatic wax, spices or essential oil) is being heated, the emitted aromatic vapor particles go through the first pores 1263 of the fiber fixation layer 1261 and then the second pores 1264 of the microporous layer 1262 toward the outside open air; the effect produced by the breathing film 126 is same is same as described above.

In one embodiment of the present invention, the breathable aperture of the first pores 1263 of the fiber fixation layer 1261 is greater than or equal to the breathable aperture of the second pores 1264 of the microporous layer 1262, facilitating passing of the generated aromatic vapor molecules through the breathing film 126, i.e., the ventilatory capacity per unit area of the first pores 1263 of the fiber fixation layer 1261 is greater than or equal to the ventilatory capacity per unit area of the second pores 1264 of the microporous layer 1262, facilitating passing of released aromatic vapor molecules through the breathing film 126.

In one embodiment of the present invention, the porosity of the first pores 1263 of the fiber fixation layer 1261 and the porosity of the second pores 1264 of the microporous layer 1262 are unequal.

The hollow heating element 16 can be a positive temperature coefficient (PTC), cement resistor or resistor type heating element.

Although particular embodiments of the present invention have been described in detail for purposes of illustration, various modifications and enhancements may be made without departing from the spirit and scope of the invention. Accordingly, the invention is not to be limited except as by the appended claims.

What the invention claimed is:

1. An aromatherapy device comprising an aroma diffuser, said aroma diffuser comprising a connection device, a fixing device set, a hollow heating element, a thermal conduction device, a shell and a base, said shell having a top opening and an opposing bottom opening, said base being fastened to said bottom opening of said shell, said connection device being mounted inside said shell and having one end thereof facing toward said top opening and fastened to a bottom center of said thermal conduction device and an opposing end thereof connected to said base inside said shell, said hollow heating element surrounding said connection device, said fixing device set supporting a bottom side of said hollow heating element, said hollow heating element having an opposing top side abutted against a bottom side of said thermal conduction device.

2. The aromatherapy device as claimed in claim 1, wherein said aroma diffuser is adapted for selectively heating a disposable multilayer breathing film-based aroma capsule or a detachable aromatic substance container, said aromatic substance container being used for filling an aromatic substance, said disposable multilayer breathing film-based aroma capsule or said aromatic substance container being to be placed on said thermal conduction device of said aroma diffuser.

3. The aromatherapy device as claimed in claim 1, wherein said hollow heating element is selectively an annular heating element, a hollow plum-shaped heating element, a hollow rectangular heating element, a hollow triangular heating element, a hollow diamond-shaped heating element, or a hollow irregular ring-shaped heating element.

4. The aromatherapy device as claimed in claim 3, wherein said hollow heating element comprises an inner bottom flange inwardly extended from a bottom surface thereof, and a connection holes surrounded by said inner bottom flange; said connection device is inserted through said connection hole and fastened to the bottom center of said thermal conduction device.

5. The aromatherapy device as claimed in claim 2, wherein said hollow heating element is selectively an annular heating element, a hollow plum-shaped heating element, a hollow rectangular heating element, a hollow triangular heating element, a hollow diamond-shaped heating element, or a hollow irregular ring-shaped heating element.

6. The aromatherapy device as claimed in claim 5, wherein said hollow heating element comprises an inner bottom flange inwardly extended from a bottom surface thereof, and a connection holes surrounded by said inner bottom flange; said connection device is inserted through said connection hole and fastened to the bottom center of said thermal conduction device.

7. The aromatherapy device as claimed in claim 1, wherein said thermal conduction device is selectively a metal sheet, a ceramic sheet, a metal container, or a ceramic container.

8. The aromatherapy device as claimed in claim 1, wherein said shell is selected from light-transmissive or semi-light-transmissive materials, having an inner flange formed on an inner side thereof; said thermal conduction device is mounted on a top side of said inner flange of said shell; said aroma diffuser further comprises a light-emitting device mounted inside said shell.

9. The aromatherapy device as claimed in claim 1, wherein said base comprises a stud, said stud having a hole in the center; said connection device is fastened to the hole of said stud of said base; said shell is provided with a plurality of light holes and mounted with a light-emitting device.

10. The aromatherapy device as claimed in claim 9, wherein said shell is provided a diffuser for creating a halo effect, said diffuser for creating a halo effect being fastened to said light hole.

11. The aromatherapy device as claimed in claim 1, wherein said connection device is a screw rod; said fixing device set comprises an anti-loosening nut and a washer, said washer defining therein a hole, the hole of said washer being mounted onto said screw rod of said connection device, said anti-loosening nut comprising a plurality of friction spines located on a top surface thereof, said anti-loosening nut being threaded onto said screw rod of said connection device and stopped against said washer.

12. The aromatherapy device as claimed in claim 1, wherein said multilayer breathing film-based aroma capsule, comprising:
a heat-transfer container defining a top opening;
an aromatic substance placed in said heat-transfer container; and
a breathing film having a breathable function and being bonded to said heat-transfer container over said top opening to keep said aromatic substance in said heat-transfer container, said breathing film comprising a fiber fixation layer defining therein a plurality of first pores and microporous layer defining therein a plurality of second pores and bonded to said microporous layer.

13. The aromatherapy device as claimed in claim 12, wherein said microporous layer is made out of thermoplastic elastomer with said second pores defined therein; said fiber fixation layer is made out of polyester with said first pores defined therein.

14. The aromatherapy device as claimed in claim 13, wherein said polyester is selected from the group consisting of Tetoron and polyethylene terephthalate (PET); said thermoplastic elastomer is thermoplastic polyurethane (TPU).

15. The aromatherapy device as claimed in claim 14, wherein said heat-transfer container is selected from the material group consisting of plastic, polyester plastic, poly chloro terephthalate glycol (PCTG), polyethylene terephthalate (PET), propylthiouracil (PTU) and polypropylene (PP).

16. The aromatherapy device as claimed in claim 15, wherein said heat-transfer container comprises a rim extended around said top opening; said breathing film is bonded to said rim of said heat-transfer container around said top opening.

17. The aromatherapy device as claimed in claim 16, wherein said thermoplastic polyurethane (TPU) of said microporous layer of said breathing film is bonded to said rim of said heat-transfer container around said top opening.

18. The aromatherapy device as claimed in claim 12, wherein said aromatic substance is selected from the group consisting of aromatic wax, balsam, essential oil, spices and sesame oil mixture.

19. The aromatherapy device as claimed in claim 12, wherein the breathable aperture of said first pores is greater than the breathable aperture of said second pores.

20. The aromatherapy device as claimed in claim 12, wherein the porosity of said first pores and the porosity of said second pores are different.
